(19) United States
(12) Patent Application Publication
(10) Pub. No.: US 2003/0012797 A1
(43) Pub. Date: Jan. 16, 2003

(54) SOFT CAPSULE

(76) Inventors: TOSHIMITSU ISHIKAWA, SHIZUOKA (JP); NOBUYUKI WADA, SHIZUOKA (JP); FUTAO KAWAGUCHI, SHIZUOKA (JP); KOJI KAJIMA, SHIZUOKA (JP)

Correspondence Address: WENDEROTH LIND & PONACK LLP, 2033 K STREET NW, SUITE 800, WASHINGTON, DC, 20006

(21) Appl. No.: 09/393,168

(22) Filed: Sep. 10, 1999

(30) Foreign Application Priority Data
Sep. 11, 1998 (JP) 10-258981

(51) Int. Cl. A61K 47/00

(52) U.S. Cl. 424/400

(57) ABSTRACT

A soft capsule capable being substantially free of or having a minimum content of a fat and oil material and an emulsifier in an encapsulated liquid or medicinal liquid. The soft capsule contains a dietary fiber in an amount of 5 to 90% by weight based on a whole composition of the medicinal liquid.

### BACKGROUND OF THE INVENTION

This invention relates to a soft capsule, and more particularly to a soft capsule for foods, pharmaceuticals, cosmetics or the like.

Conventionally, a capsule has been commercially available in the art which is constructed in such a manner that a fat and oil material, an oil-soluble material, an oil-soluble perfume, and/or the like are charged or encapsulated in an encapsulating material made of gelatin. Such a capsule permits any ingredient in any desired amount to be readily provided or each dose to be handy to carry. Also, the capsule effectively prevents contact of an encapsulated ingredient with an ambient atmosphere, to thereby ensure stability of the ingredient.

A medicinal liquid which is an ingredient or material encapsulated in a soft capsule is constituted of a first medicinal liquid ingredient of a fat and oil material and a second medicinal liquid ingredient obtained by adding an effective component extract and/or an effective component powder to a fat and oil material to prepare a mixture and stabilizing the mixture with a suitable emulsifier. Then, the medicinal liquid thus obtained is charged or encapsulated in a soft encapsulating material, resulting in the soft capsule being provided. Methods of producing soft capsules include techniques using a rotary type capsule manufacturing equipment, a seamless type capsule manufacturing equipment and a flat plate type capsule manufacturing equipment.

Unfortunately, the soft capsule manufactured by the conventional procedure causes much time to be required for preparation of the medicinal liquid or stock solution, leading to a deterioration in both workability and productivity and an increase in production cost. Also, it tends to cause excessive intake of calorie because of containing a relative large amount of fats and oils.

### SUMMARY OF THE INVENTION

The present invention has been made in view of the foregoing disadvantages of the prior art while taking notice of the fact that formulation of a dietary fiber in a prescribed amount in a medicinal liquid of a soft capsule permits the soft capsule to exhibit desired performance or characteristics.

Accordingly, it is an object of the present invention to provide a soft capsule which is completely free of such fats and oils and emulsifier or which has a minimum content of such fats and oils and emulsifier as contained in a conventional soft capsule.

It is another object of the present invention to provide a soft capsule which is capable of permitting a calorie intake due to taking of the soft capsule to be significantly restrained while ensuring concurrent intake of a dietary fiber.

It is a further object of the present invention to provide a soft capsule which is capable of being increased in productivity and reduced in manufacturing cost.

In accordance with the present invention, a soft capsule is provided. The soft capsule contains a dietary fiber in an amount of 5 to 90% by weight based on a whole composition of a medicinal liquid of the soft capsule.

In a preferred embodiment of the present invention, the dietary fiber is contained in an amount of 5 to 60% by weight based on the whole composition of the medicinal liquid.

In a preferred embodiment of the present invention, the dietary fiber is either a micronized vegetable fiber or a water-soluble soluble dietary fiber.

In a preferred embodiment of the present invention, the soft capsule is substantially free of any dispersion stabilizer.

In a preferred embodiment of the present invention, the soft capsule is substantially free of any fat and oil material or oil-soluble material.

In a preferred embodiment of the present invention, the soft capsule may further contain either a fat and oil material or an oil-soluble material in an amount of 50% or less by weight based on the whole composition of the medicinal liquid.

In a preferred embodiment of the present invention, the soft capsule may further contain a material of limited oil-solubility in an amount of 1 to 80% by weight based on the whole composition of the medicinal liquid.

In a preferred embodiment of the present invention, the soft capsule may further contain a material of limited oil-solubility in an amount of 1 to 70% by weight based on the whole composition of the medicinal liquid, wherein the fat and oil material or oil-soluble material is contained in an amount of 1 to 50% by weight based on the whole composition of the medicinal liquid.

### DETAILED DESCRIPTION OF THE PREFERRED EMBODIMENT

One of features of a soft capsule according to the present invention is that it contains a dietary fiber in an mount of 5 to 90% by weight based on a whole composition of a medicinal liquid for the soft capsule.

The dietary fiber is preferably a micronized vegetable fiber or a water-soluble dietary fiber, unlike a conventional dietary fiber. Such a dietary fiber is commercially available as a food stock at a reduced cost, resulting in being readily provided.

The dietary fiber is incorporated or contained in an amount of 5 to 90% by weight based on the whole composition of the medicinal liquid for the soft capsule. Such a content of the dietary fiber leads to preparation of a satisfactory suspended stock solution. Preferably, a content of the dietary fiber is 5 to 60% by weight. The content permits the suspended stock solution to be increased in stability.

Materials formulated in the medicinal liquid of the soft capsule include any desired powder material, extract material, fat and oil material which are conventionally formulated in foods, pharmaceuticals or cosmetics.

Powder materials of poor or limited oil-solubility and soft extract materials of poor or limited oil-solubility suitable for use in the present invention include chitosan, a water-soluble vitamin, an animal powder, a vegetable powder, a dietary fiber, an extract powder of a crude drug, an animal extract, a vegetable extract, a mineral and the like.

The powder material of limited oil-solubility, which has an average particle size sufficient to permit the material to pass through a screen of 60 to 200 meshes, and the soft extract material of limited oil-solubility, which has a water and alcohol content of 50% or less, are preferably formulated in an amount of 1 to 70% by weight based on the whole composition of the contents of the soft capsule. More preferably, they are formulated in an amount of 5 to 60% by weight.

A reduction in amount of the powder material and soft extract material of limited oil-solubility formulated facilitates manufacturing of the soft capsule, however, it brings about a disadvantage of causing an increase in intake of the soft capsule because it is required to take the materials in an amount sufficient to permit the materials to exhibit desired effects. Formulation of the materials in an amount above 50% by weight causes manufacturing of the soft capsule to be complicated and workability to be deteriorated. Also, it leads to a deterioration in stability of the suspended stock solution.

In the soft capsule of the present invention, the medicinal liquid is substantially free of any emulsifying agent. The term “substantially free” used herein indicates both a case that the medicinal liquid does not contain any emulsifying agent and a case that it contains the agent in a concentration substantially decreased as compared with the conventional soft capsule.

The soft capsule of the present invention may contain a fat and oil material and an oil-soluble material. Alternatively, it may not contain the materials. When the soft capsule of the present invention is free of the fat and oil material and oil-soluble material, the materials are fully replaced with a dietary fiber. In this instance, this causes a problem that the soft capsule exhibits no effects of the fat and oil material and oil-soluble material, however, it permits a calorie intake derived from the fat and oil material to be substantially restrained and intake of a dietary fiber to be increased. Also, the soft capsule of the present invention permits a content of the fat and oil material and oil-soluble material to be significantly reduced as compared with the conventional soft capsule, even when it contains the materials.

Also, the fat and oil material (edible fat and oil) and oil-soluble material which may be contained in the soft capsule of the present invention include animal and vegetable edible fats and oils such as borage oil, evening primrose oil, perilla oil, refined fish oil containing DHA and/or EPA, liver oil, olive oil, safflower oil, egg yolk oil and the like; as well as oil-soluble materials such as vitamin E, vitamin A, vitamin D, carotenoid and the like.

The fat and oil material and oil-soluble material are preferably formulated in an amount of 1 to 50% by weight based on the whole composition of the contents of the soft capsule of the present invention. A stock solution contained or encapsulated in the soft capsule which is suspended by formulation of the powder material of limited oil-solubility, soft extract material of limited oil-solubility, dietary fiber and oil-soluble material exhibits increased stability as compared with any suspended stock solution conventionally commercially available.

An encapsulating material of the soft capsule is generally constituted of a combination of gelatin, glycerine, sugar and the like. Other ingredients which may be incorporated in the encapsulating material include eggshell calcium, alginic acid, sodium alginate, caramel, carrageenin, starch, flavor and the like.

The suspended stock solution encapsulated in the soft capsule of the present invention may be prepared by blending the powder material/soft extract material of limited oil-solubility with the dietary fiber and the fat and oil material/oil-soluble material to form a mixture and then subjecting the mixture to agitation for 20 to 30 minutes using a high-speed agitator, such as an agitator commercially available under a trade name “HOMO JETTOR” from TOKUSHU KIKA KOGYO CO., LTD. or the like, or a high-speed grinder, resulting in the mixture being homogenized. During the treatment, the high-speed grinder or high-speed agitator is set at a rotational speed of 4000 to 6000 rpm.

The suspended stock solution thus prepared exhibits increased stability due to action of the dietary fiber as compared with the conventional stock solution employing a suspension stabilizer (emulsion stabilizer) such as beeswax, glycerine fatty ester or the like.

Also, the suspended stock solution contained in the soft capsule of the present invention permits manufacturing of the soft capsule to be highly improved and time required for the manufacturing to be substantially reduced. The soft capsule of the present invention thus manufactured effectively prevents leakage of the solution from a sealing portion of the encapsulating material and enhances stability with time, leading to an increase in quality.

As can be seen form the foregoing, the soft capsule of the present invention permits a content of the less oil-soluble powder material and less oil-soluble soft extract material therein to be significantly increased. Also, it may be substantially free of any fat and oil material and emulsifier or minimize a content thereof.

Further, the soft capsule of the present invention permits the material of limited oil-solubility and powder material of limited oil-solubility containing water in an increased concentration to be highly dispersed therein, resulting in its being increased in stability with time.

The invention will be understood more readily with reference to the following examples; however, the examples are intended to illustrate the invention and are not to be construed to limit the scope of the invention.

Prior to description of the examples, test procedures employed in the examples will be described.

Test procedure 1: Test on dispersion stability of suspended stock solution

The suspended stock solution prepared by formulation is charged in a transparent vessel made of glass and formed to have a diameter of about 1 cm and a depth of about 10 cm. Then it is stored in a constant temperature bath at a temperature of 50° C. while being capped. Dispersion stability of the suspended stock solution is observed after 24 hours, 48 hours, 72 hours, one month and three months. Also, the suspended stock solution is subjected to centrifuging at a speed of 3000 rpm for 1 minute while being charged in a test tube (Spitzglas) of a centrifuge, resulting in dispersion stability of the suspended stock solution being observed.

Test procedure 2: Test on stability of soft capsule having suspended stock solution encapsulated therein with time

The soft capsule having the suspended stock solution encapsulated therein is manufactured. About 100 such soft capsules are charged in a glass vessel and stored in a thermohygrostat at a temperature of 40° C. and a humidity of 75% while being tightly capped, resulting in an appearance of the soft capsules, smell thereof, tone thereof, suspension thereof, leakage of liquid therefrom and the like being observed after one month, three months and six months.

### EXAMPLE 1

A suspended stock solution having a composition shown in Table 1 was prepared. A dietary fiber was added to a raw royal jelly to prepare a mixture, which was then subjected to homogenizing at a speed of 4000 rpm for 15 minutes by means of a high-speed agitator “HOMO JETTOR” while gradually increasing the speed to a level of 4000 rpm. The temperature was kept at about 40° C.

The thus-prepared suspended stock solution was subjected to degassing at a pressure of 720 mmHg. Then, the degassed suspended stock solution was treated by means of a rotary-type soft capsule manufacturing equipment, resulting in a suppository-type soft capsule being provided.

### COMPARATIVE EXAMPLE 1

Beeswax and glycerine fatty ester melted by heating so as to act as a dispersion stabilizer were added to a fat and oil material constituted by safflower oil to prepare a mixture, which was then subjected to homogenizing at a temperature of 65+2° C. and a speed of 6000 rpm for about 20 minutes by means of a high-speed agitator “HOMO JETTOR” while gradually increasing a speed of the agitator to 6000 rpm, resulting in an intermediate suspended stock solution homogenized being obtained.

Thereafter, the intermediate suspended stock solution was cooled at a room temperature to 40° C. or less, to thereby be rendered semisolid. Then, a raw royal jelly constituting a material of limited oil-solubility was added to the semisolid intermediate suspended stock solution while being kneaded into the intermediate suspended stock solution. This resulted in the intermediate suspended stock solution being ready to be treated by means of a high-speed agitator “HOMO JETTOR”. Then, the agitator was gradually increased in rotational speed, to thereby permit the intermediate stock solution to be subjected to homogenizing at a speed of 6000 rpm for about 30 minutes, resulting in a suspended stock solution being prepared.

The suspended stock solution thus prepared was subjected to degassing at a pressure of 720 mmHg. Then, the degassed suspended stock solution was treated by means of a rotary-type soft capsule manufacturing equipment, resulting in a suppository-type soft capsule being provided.

TABLE 1

| | |
|---|---|
| | Comparative |
| Example 1 | Example 1 |

Composition

| | | | | |
|---|---|---|---|---|
| Fat and oil material: | | | | |
| Safflower oil | | | 168 | mg |
| Material of limited oil- | | | | |
| solubility: | | | | |
| Raw royal jelly | 180 | mg | 90 | mg |
| Dispersion Stabilizer: | | | | |
| Beeswax | | | 21 | mg |
| Glycerine fatty ester | | | 21 | mg |
| Dietary fiber | 120 | mg | | |
| Total | 300 | mg | 300 | mg |

| | | |
|---|---|---|
| Fat and oil material (%) | | 56 |
| Material of limited oil- | 60 | 30 |
| solubility (%) | | |
| Dietary fiber (%) | 40 | |
| Dispersion stabilizer (%) | | 14 |
| Total (%) | 100 | 100 |

Stability of suspended stock solution

| | | |
|---|---|---|
| Temperature of 50° C. | No separation | Slight separation |
| Centrifuging | No separation | Separation |
| Workability | Good | Poor |
| Liquid leakage | No | Yes |
| Stability with time | Good | Nasty smell |
| (Appearance, smell, tone) | | |
| Suspension | No separation | Tendency |
| | | to separate |

Example 1 and Comparative Example 1 clearly indicate that containing of the raw royal jelly in an increased amount causes much difficulty.

A content of a raw royal jelly in the conventional soft capsule is generally up to 30%. However, Example 1 indicates that the soft capsule of the present invention permits the raw royal jelly to be contained in an amount as high as 60% by adding the dietary fiber. Also, Example 1 indicates that the present invention permits the suspended stock solution increased in suspension stability to be prepared without incorporating any fat and oil material and any suspension stabilizer such as beeswax, glycerine fatty ester or the like in the suspended stock solution. Further, it indicates that the present invention permits the suspended stock solution to be encapsulated in the soft capsule while being significantly increased in both stability with time and quality.

### EXAMPLE 2

A suspended stock solution having a composition shown in Table 2 was prepared. A fat and oil material and a dietary fiber were homogenized together to prepare a homogenized mixture in a manner similar to that in Example 1 and then a material of limited oil-solubility was added to the mixture, resulting in the suspended stock solution being provided. Then, a soft capsule of the present invention was manufactured in which the thus-prepared suspended stock solution was encapsulated. Though a content of the material of limited oil-solubility exceeded 50% of the composition of the medicinal liquid, the soft capsule exhibited improved workability, increased sealing properties, increased stability with time and enhanced suspension stability and was kept from any liquid leakage.

TABLE 2

Example 2

Composition

| | | |
|---|---|---|
| Fat and oil material: | | |
| Vegetable oil | 15 | mg |
| Material of limited oil-solubility: | | |
| Gimnema sylvesta powder | 210 | mg |
| Dietary fiber | 30 | mg |
| Water | 45 | mg |
| Total | 300 | mg |

| | |
|---|---|
| Fat and oil material | 5% |
| Material of limited oil-solubility | 70% |
| Dietary fiber | 10% |
| Water | 15% |
| Total | 100% |

Suspension stability

| | |
|---|---|
| Temperature of 50° C. | No separation |
| Centrifuging | No separation |
| Workability | Good |
| Liquid leakage | No |
| Stability with time | Good |
| (Appearance, smell, tone) | |
| Suspension | No separation |

While the present invention has been described with a certain degree of particularity, obvious modifications and variations are possible in light of the above teachings. It is therefore to be understood that within the scope of the appended claims, the invention may be practiced otherwise than as specifically described.

What is claimed is:

1. A soft capsule comprising a dietary fiber in an amount of 5 to 90% by weight based on a whole composition of a medicinal liquid of said soft capsule.

2. A soft capsule as defined in claim 1, wherein said dietary fiber is contained in an amount of 5 to 60% by weight based on the whole composition of said medicinal liquid.

3. A soft capsule as defined in claim 1, wherein said dietary fiber is either a micronized vegetable fiber or a water-soluble dietary fiber.

4. A soft capsule as defined in claim 2, wherein said dietary fiber is either a micronized vegetable fiber or a water-soluble dietary fiber.

5. A soft capsule as defined in claim 1, wherein said soft capsule is substantially free of any dispersion stabilizer.

6. A soft capsule as defined in claim 2, wherein said soft capsule is substantially free of any dispersion stabilizer.

7. A soft capsule as defined in claim 3, wherein said soft capsule is substantially free of any dispersion stabilizer.

8. A soft capsule as defined in claim 4, wherein said soft capsule is substantially free of any dispersion stabilizer.

9. A soft capsule as defined in claim 1, wherein said soft capsule is substantially free of any fat and oil material or oil-soluble material.

10. A soft capsule as defined in claim 2, wherein said soft capsule is substantially free of any fat and oil material or oil-soluble material.

11. A soft capsule as defined in claim 3, wherein said soft capsule is substantially free of any fat and oil material or oil-soluble material.

12. A soft capsule as defined in claim 5, wherein said soft capsule is substantially free of any fat and oil material or oil-soluble material.

13. A soft capsule as defined in claim 1, further comprising either a fat and oil material or an oil-soluble material in an amount of 50% or less by weight based on the whole composition of said medicinal liquid.

14. A soft capsule as defined in claim 2, further comprising either a fat and oil material or an oil-soluble material in an amount of 50% or less by weight based on the whole composition of said medicinal liquid.

15. A soft capsule as defined in claim 3, further comprising either a fat and oil material or an oil-soluble material in an amount of 50% or less by weight based on the whole composition of said medicinal liquid.

16. A soft capsule as defined in claim 5, further comprising either a fat and oil material or an oil-soluble material in an amount of 50% or less by weight based on the whole composition of said medicinal liquid.

17. A soft capsule as defined in claim 9, further comprising a material of limited oil-solubility in an amount of 1 to 80% by weight based on the whole composition of said medicinal liquid.

18. A soft capsule as defined in claim 14, further comprising a material of limited oil-solubility in an amount of 1 to 70% by weight based on the whole composition of said medicinal liquid;
said fat and oil material or oil-soluble material being contained in an amount of 1 to 50% by weight based on the whole composition of said medicinal liquid.

19. A soft capsule consisting essentially of:
a dietary fiber in an amount of 5 to 90% by weight based on a whole composition of a medicinal liquid of said soft capsule; and
a material of limited oil-solubility in an amount of 1 to 80% by weight based on the whole composition of said medicinal liquid.

20. A soft capsule consisting essentially of:
a dietary fiber in an amount of 5 to 60% by weight based on a whole composition of a medicinal liquid of said soft capsule;
a material of limited oil-solubility in an amount of 1 to 70% by weight based on the whole composition of said medicinal liquid; and
either a fat and oil material or an oil-soluble material in an amount of 1 to 50% by weight based on the whole composition of said medicinal liquid.
